SEQUENCE LISTING (15 sequences)

SEQ ID NO: 1 - protein, 7 aa, Artificial Sequence
  CDR1 of hsd2Ab
STFSEDA

SEQ ID NO: 2 - protein, 7 aa, Artificial Sequence
  CDR2 of hsd2Ab
GFANPHS

SEQ ID NO: 3 - protein, 15 aa, Artificial Sequence
  ASHKKTRAPAATFET
ASHKKTRAPAATFET

SEQ ID NO: 4 - protein, 28 aa, Artificial Sequence
  FR1 of hsd2Ab
MAEVQLQASGGGFVQPGGSLRLSCAASG

SEQ ID NO: 5 - protein, 19 aa, Artificial Sequence
  FR2 of hsd2Ab
MGWFRQAPGKEREFVSAIS

SEQ ID NO: 6 - protein, 39 aa, Artificial Sequence
  FR3 of hsd2Ab
YYADSVKGRFTISRDNSKNTVYLQMNSLRAEDTATYYCA

SEQ ID NO: 7 - protein, 12 aa, Artificial Sequence
  FR4 of hsd2Ab
YWGQGTQVTVSS

SEQ ID NO: 8 - protein, 127 aa, Artificial Sequence
  full sequence of G1N1 VHH
MAEVQLQASGGGFVQPGGSLRLSCAASGSTFSEDAMGWFRQAPGKEREFVSAISGFANPHSYYADSVKGRFTISRDNSKNTVYLQMNSLRAEDTATYYCAASHKKTRAPAATFETYWGQGTQVTVSS

SEQ ID NO: 9 - protein, 124 aa, Artificial Sequence
  G1N2 VHH
MAEVQLQASGGGFVQPGGSLRLSCAASGSTYFHTAMGWFRQAPGKEREFVSAISGFANPHRYYASSVKGRFTISRDNSKNTVYLQMNSLRAEDTATYYCANLISTNRGWLMGYWCQGTQVTVSS

SEQ ID NO: 10 - protein, 135 aa, Homo sapiens
MACGLVASNLNLKPGECLRVRGEVAPDAKSFVLNLGKDSNNLCLHFNPRFNAHGDANTIVCNSKDGGAWGTEQREAVFPFQPGSVAEVCITFDQANLTVKLPDGYEFKFPNRLNLEAINYMAADGDFKIKCVAFD

SEQ ID NO: 11 - protein, 139 aa, Homo sapiens
MSSLPVPYKLPVSLSVGSCVIIKGTPIHSFINDPQLQVDFYTDMDEDSDIAFRFRVHFGNHVVMNRREFGIWMLEETTDYVPFEDGKQFELCIYVHYNEYEIKVNGIRIYGFVHRIPPSFVKMVQVSRDISLTSVCVCN

SEQ ID NO: 12 - protein, 142 aa, Homo sapiens
MSFLTVPYKLPVSLSVGSCVIIKGTLIDSSINEPQLQVDFYTEMNEDSEIAFHLRVHLGRRVVMNSREFGIWMLEENLHYVPFEDGKPFDLRIYVCLNEYEVKVNGEYIYAFVHRIPPSYVKMIQVWRDVSLDSVLVNNGRR

SEQ ID NO: 13 - protein, 136 aa, Homo sapiens
MSNVPHKSSLPEGIRPGTVLRIRGLVPPNASRFHVNLLCGEEQGSDAALHFNPRLDTSEVVFNSKEQGSWGREERGPGVPFQRGQPFEVLIIASDDGFKAVVGDAQYHHFRHRLPLARVRLVEVGGDVQLDSVRIF

SEQ ID NO: 14 - protein, 132 aa, Homo sapiens
MTGELEVKNMDMKPGSTLKITGSIADGTDGFVINLGQGTDKLNLHFNPRFSESTIVCNSLDGSNWGQEQREDHLCFSPGSEVKFTVTFESDKFKVKLPDGHELTFPNRLGHSHLSYLSVRGGFNMSSFKLKE

SEQ ID NO: 15 - protein, 135 aa, Mus musculus
MACGLVASNLNLKPGECLKVRGEVASDAKSFVLNLGKDSNNLCLHFNPRFNAHGDANTIVCNTKEDGTWGTEHREPAFPFQPGSITEVCITFDQADLTIKLPDGHEFKFPNRLNMEAINYMAADGDFKIKCVAFE

(19) United States
(12) Patent Application Publication
(10) Pub. No.: US 2024/0228627 A1
(43) Pub. Date: Jul. 11, 2024

(54) GALECTIN-1-SPECIFIC MONOVALENT ANTIBODIES AND USES THEREOF

(71) Applicant: INSTITUT NATIONAL DE LA RECHERCHE SCIENTIFIQUE, Québec, Québec (CA)

(72) Inventors: Yves St-Pierre, Canmore, Alberta (CA); Nicolas Doucet, Laval, Québec (CA); David Chatenet, Lorraine, Québec (CA)

(21) Appl. No.: 18/558,963

(22) PCT Filed: May 3, 2022
(86) PCT No.: PCT/CA2022/050688

§ 371 Date: Nov. 3, 2023

Related U.S. Application Data
(60) Provisional application No. 63/201,569, filed on May 5, 2021.

(51) Int. Cl. C07K 16/28; A61K 47/68; A61K 51/10

(52) U.S. Cl. CPC C07K 16/2851; A61K 47/6849; A61K 51/1027; C07K 2317/22; C07K 2317/569

(57) ABSTRACT

Monovalent antibodies such as nanobodies that are specific for galectin-1 are described. These monovalent antibodies are able to interfere with the activity of galectin-1, and thus may be used for the treatment of diseases associated with dysregulated galectin-1 expression and/or activity, such as certain types of cancers, as well as conditions associated with pathological angiogenesis or fibrosis.

### CROSS REFERENCE TO RELATED APPLICATIONS

The present application claims the benefit of U.S. provisional patent application No. 63/201,569 filed on May 5, 2021, which is incorporated herein by reference in its entirety.

### TECHNICAL FIELD

The present invention generally relates to galectins, and more specifically to the detection of galectin-1 (GAL-1) and to the modulation of GAL-1 activity.

### BACKGROUND ART

In the field of biology, carbohydrates are defined as organic compounds composed of carbon, hydrogen, and oxygen that are organized into ring structures. The analysis of these structures and their functions has led to a new field of biology called “glycobiology.” In the biomedical sciences, glycobiology is rapidly emerging as an integral part of complex biological processes. Evidence suggesting that the interactions between lectins and their ligands play a major role in the different steps of cancer progression has accumulated at a rapid pace and has gained the attention of several oncologists [reviewed by Pinho and Reis, 2015]. This is particularly true for galectin family members because changes in their expression levels correlate with alterations in cancer cell growth, apoptosis, and cell-cell and cell-matrix interactions [reviewed by Liu and Rabinovich, 2005].

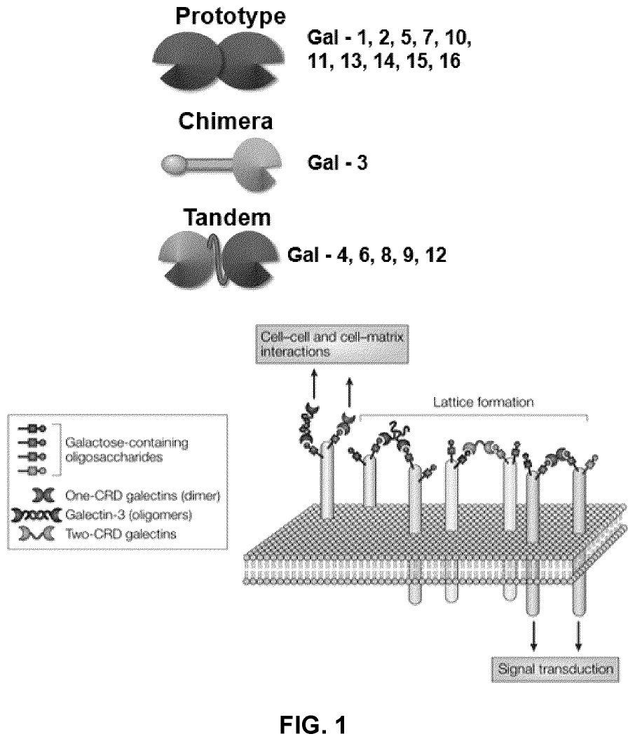

Galectins (GAL) are multifunctional proteins that belong to the animal lectin family. All galectins share similar binding affinities to β-galactosides and display some sequence and structural similarities among their carbohydrate-recognition domains (CRDs) [Barondes et al., 1994]. Galectins can be found in the cytoplasm, the nucleus, or can be secreted by the cell, a mechanism which occurs via a non-classical secretory pathway. The distribution of galectins is tissue specific, and their expression is developmentally regulated [Cummings and Liu, 2009]. In mammals, 19 different members of this family have been identified, with 13 of them being expressed in humans. GAL-5, -6, -11, -15, -16, -19, and -20 are not found in human (FIG. 1). Galectins are divided into three sub-groups according to their structure: prototypic galectins containing one CRD (GAL-1, -2, -5, -7, -10, -11, -13, -14, -15, -16, -17, -19, and -20), tandem-repeat galectins containing two covalently linked CRDs (GAL-4, -6, -8, -9 and -12) and chimera-type galectins containing multiple CRDs linked by their amino-terminal domain (GAL-3).

Although galectins are involved in various physiological processes, they are best known for their immunoregulatory roles when they are released either passively from dead cells or actively via non-classical secretion pathways [Liu et al., 2008; Rabinovich and Toscano, 2009]. This was documented first in a landmark paper published by the group of Linda Baum showing that galectin-1 (GAL-1) can induce apoptosis of activated T cells [Perillo et al., 1997]. The immunosuppressive role of galectins is also highlighted by studies showing that administration of recombinant prototypic GAL-1 prevents disease progression in patients with autoimmune disorders [Sunblad at al., 2017; Allo et al., 2020]. Since the initial discovery by the group of Linda Baum, the immunoregulatory role of galectins has been extended to most if not all members of the family. For example, several members of the galectin family, such as GAL-13, GAL-14, and GAL-16, contribute to the generation of an immune-privileged environment at the maternal-fetal interface [Than et al., 2014].

Expression of galectins undergoes strict mechanisms of regulation. This is not surprising given their important role in the regulation of the immune response. In cancer cells, however, the expression of galectins often reaches abnormally high levels, favoring an increase of galectin concentrations in the extracellular milieu [Grosset et al., 2016; Labrie at al., 2017]. Such accumulation of galectins creates systemic and local immunosuppressive microenvironments that promotes cancer progression and metastasis, which represents a significant obstacle to cancer immunotherapy [reviewed by Rodriguez et al., 2018 and more recently by Jin et al., 2021]. This is especially true for GAL-1, for which the immunosuppressive role has been revealed in a relatively large number of cancer subtypes, including solid tumors [Cedeno-Laurent at al., 2012; Salatino et al., 2013; Tang et al., 2015; Wu et al., 2015; Batzke et al., 2018; Chen et al., 2019]. This immunosuppressive function of galectins is mediated upon binding of extracellular galectins to cell surface glycoreceptors expressed on immune cells. On activated T cells, this triggers a cascade of signaling events that leads to apoptosis of cancer-killing T cells. Consequently, GAL-1 has been now recognized as a significant obstacle to successful cancer immunotherapy [Chakraborty and Dimitroff, 2019; Nambiar et al., 2019]. This has been well established in several types of cancer. In lymphoma, for example, secretion of GAL-1 is responsible for resistance to anti-CD20 immunotherapy [Lykken et al., 2016]. Targeting GAL-1 by genetic knockdown has also been shown to improve immunotherapy against glioblastoma [Verschuere et al., 2014; Chen at al., 2019]. Neutralization of galectins by intranasal delivery of siRNA was also shown to stimulate immune activation within the tumor microenvironment, thereby increasing the efficiency of immune-checkpoint inhibitors (PD-1 blocking) [Van Woensel et al., 2017].

Given their important role in cancer and other diseases, considerable efforts have been directed towards the development of galectin inhibitors. Despite almost two decades of research, however, the development of effective and specific galectin-1 antagonists has met limited success [Blanchard et al., 2016]. In most cases, these inhibitors are high molecular weight, naturally occurring polysaccharides that are used to block the binding of extracellular galectins to carbohydrate structures on cell surface receptors. Yet, the greatest challenge to these glycan-binding site (GBS) targeting drugs is achieving high selectivity. This is a challenging task considering the striking structural similarity between the GBS of multiple homologous galectins [Cummings and Liu, 2009]. Such structural similarity also complicates the use of conventional blocking antibodies, a logical strategy to generate inhibitors that would specifically target de GBS of galectins. Moreover, the use conventional antibodies for the treatment of solid tumors is a challenging task given their poor tissue penetration [Cruz and Kayser, 2019].

### SUMMARY

The present disclosure provides the following items 1 to 72:

1. A monovalent antibody that specifically binds to human galectin-1 (hGAL-1) and inhibits its activity, wherein the monovalent antibody comprises the following combination of complementarity determining regions (CDRs): a CDR1 comprising an amino acid sequence having at least 80% identity with the sequence STFSEDA (SEQ ID NO:1); a CDR2 comprising an amino acid sequence having at least 80% identity with the sequence GFANPHS (SEQ ID NO:2); and a CDR3 comprising an amino acid sequence having at least 80% identity with the sequence ASHKKTRAPAATFET (SEQ ID NO:3).

2. The monovalent antibody of item 1, which comprises one of the following combinations of CDRs: a CDR1 comprising an amino acid sequence having at least 90% identity with the sequence STFSEDA (SEQ ID NO:1); a CDR2 comprising an amino acid sequence having at least 90% identity with the sequence GFANPHS (SEQ ID NO:2); and a CDR3 comprising an amino acid sequence having at least 90% identity with the sequence ASHKKTRAPAATFET (SEQ ID NO:3).

3. The monovalent antibody of item 1, which comprises one of the following combinations of CDRs: a CDR1 comprising the sequence STFSEDA (SEQ ID NO:1); a CDR2 comprising the sequence GFANPHS (SEQ ID NO:2); and a CDR3 comprising the sequence ASHKKTRAPAATFET (SEQ ID NO:3).

4. The monovalent antibody of any one of items 1 to 3, wherein the monovalent antibody is a single-domain antibody.

5. The monovalent antibody of any one of items 1 to 4, which comprises:
(i) a framework region (FR) 1 comprising an amino acid sequence having at least 50% identity with the sequence MAEVQLQASGGGFVQPGGSLRLSCAASG (SEQ ID NO:4);
(ii) a FR2 comprising an amino acid sequence having at least 50% identity with the sequence MGWFRQAPGKEREFVSAIS (SEQ ID NO:5);
(iii) a FR3 comprising or consisting of an amino acid sequence having at least 50% identity with the sequence YYADSVKGRFTISRDNSKNTVYLQMNSLRAEDTATYYCA (SEQ ID NO:6);
(iv) a FR4 comprising an amino acid sequence having at least 50% identity with the sequence YWGQGTQVTVSS (SEQ ID NO:7); or
(v) any combination of (i) to (iv).

6. The monovalent antibody of item 5, which comprises:
(i) a FR1 comprising an amino acid sequence having at least 90% identity with the sequence MAEVQLQASGGGFVQPGGSLRLSCAASG (SEQ ID NO:4);
(ii) a FR2 comprising an amino acid sequence having at least 90% identity with the sequence MGWFRQAPGKEREFVSAIS (SEQ ID NO:5);
(iii) a FR3 comprising or consisting of an amino acid sequence having at least 90% identity with the sequence YYADSVKGRFTISRDNSKNTVYLQMNSLRAEDTATYYCA (SEQ ID NO:6);
(iv) a FR4 comprising an amino acid sequence having at least 90% identity with the sequence YWGQGTQVTVSS (SEQ ID NO:7); or
(v) any combination of (i) to (iv).

7. The monovalent antibody of item 6, which comprises:

(i) a FR1 comprising the sequence
(SEQ ID NO: 4)
MAEVQLQASGGGFVQPGGSLRLSCAASG;
(ii) a FR2 comprising the sequence
(SEQ ID NO: 5)
MGWFRQAPGKEREFVSAIS;
(iii) a FR3 comprising the sequence
(SEQ ID NO: 6)
YYADSVKGRFTISRDNSKNTVYLQMNSLRAEDTATYYCA;

(iv) a FR4 comprising the sequence YWGQGTQVTVSS (SEQ ID NO:7); or
(v) any combination of (i) to (iv).

8. The monovalent antibody of any one of items 1 to 7, comprising an amino acid sequence having at least 80% identity with the sequence:

(SEQ ID NO: 8)
MAEVQLQASGGGFVQPGGSLRLSCAASGSTFSEDAMGWFRQAPGKEREF
VSAISGFANPHSYYADSVKGRFTISRDNSKNTVYLQMNSLRAEDTATYY
CAASHKKTRAPAATFETYWGQGTQVTVSS.

9. The monovalent antibody of item 8, comprising an amino acid sequence having at least 90% identity with the sequence set forth in item 8.

10. The monovalent antibody of item 9, comprising the amino acid sequence set forth in item 8.

11. The monovalent antibody according to any one of items 1 to 10, wherein said monovalent antibody is fused to at least one antibody constant domain, or a fragment thereof.

12. The monovalent antibody according to item 11, wherein the at least one antibody constant domain or fragment thereof comprises a Fragment crystallizable (Fc) region.

13. The monovalent antibody according to item 12, wherein the Fc fragment comprises a CH2 domain and CH3 domain of a human antibody.

14. The monovalent antibody of any one of items 1 to 13, wherein said antibody is conjugated to a label, a nanoparticle, a drug, a peptide, a nucleic acid, a toxin, an enzyme, a radioisotope, or a half-life extending moiety.

15. A nucleic acid comprising a nucleotide sequence encoding the monovalent antibody defined in any one of items 1 to 14.

16. The nucleic acid of item 15, which is in the form of mRNA.

17. The nucleic acid of item 15 or 16, which is encapsulated into lipid vesicles.

18. A vector comprising the nucleic acid of item 15.

19. A cell comprising the nucleic acid of any one of items 15 to 17 or the vector of item 18.

20. A pharmaceutical composition comprising the monovalent antibody defined in any one of items 1 to 14, the nucleic acid of any one of items 15 to 17 and one or more pharmaceutically acceptable carriers, excipient, and/or diluents.

21. A method for binding human galectin-1 (hGAL-1) comprising contacting said hGAL-1 with the monovalent antibody of any one of items 1 to 14 or the composition of item 20.

22. The method of item 19, wherein said hGAL-1 is expressed at the surface of a cell.

23. A method for inhibiting galectin-1-mediated apoptosis in a cell, said method comprising contacting said cell with an effective amount of the monovalent antibody of any one of items 1 to 14, or the composition of item 20.

24. The method of item 23, wherein said cell is an immune cell.

25. The method of item 24, wherein said immune cell is a T lymphocyte.

26. A method for inhibiting the activity of human galectin-1 (hGAL-1) comprising contacting said (hGAL-1) with the monovalent antibody of any one of items 1 to 14, the nucleic acid of any one of items 15 to 17, or the composition of item 20.

27. The method of item 26, wherein said hGAL-1 is expressed at the surface of a cell.

28. A method for treating a galectin-1-expressing cancer in a subject, said method comprising administering to said subject an effective amount of the monovalent antibody of any one of items to 14, the nucleic acid of any one of items 15 to 17, or the composition of item 20.

29. The method of item 28, wherein the cancer is of epithelial origin.

30. The method of item 28 or 29, wherein the cancer is a breast cancer, a melanoma, an ovarian cancer or a lymphoma.

31. The method of any one of items 28 to 30, wherein said monovalent antibody, nucleic acid or composition is administered in combination with a second anti-tumoral agent.

32. A method for treating a disease or condition associated with pathological neovascularization or angiogenesis in a subject comprising administering to said subject an effective amount of the monovalent antibody of any one of items 1 to 14, the nucleic acid of any one of items 15 to 17, or the composition of item 20.

33. A method for inhibiting tissue or organ fibrosis in a subject comprising administering to said subject an effective amount of the monovalent antibody of any one of items 1 to 14, the nucleic acid of any one of items 15 to 17, or the composition of item 20.

34. The method of any one of items 28 to 33, wherein the subject is a human subject.

35. The monovalent antibody of any one of items 1 to 14, or the composition of item 20, for use in binding human galectin-1 (hGAL-1).

36. The monovalent antibody or composition for use according to item 35, wherein said hGAL-1 is expressed at the surface of a cell.

37. The monovalent antibody of any one of items 1 to 14, or the composition of item 20, for use in inhibiting galectin-1-mediated apoptosis in a cell.

38. The monovalent antibody or composition for use according to item 37, wherein said cell is an immune cell.

39. The monovalent antibody or composition for use according to item 38, wherein said immune cell is a T lymphocyte.

40. The monovalent antibody of any one of items 1 to 14, the nucleic acid of any one of items to 17, or the composition of item 20, for use in inhibiting the activity of human galectin-1 (hGAL-1).

41. The monovalent antibody, nucleic acid or composition for use according to item 40, wherein said hGAL-1 is expressed at the surface of a cell.

42. The monovalent antibody of any one of items 1 to 14, the nucleic acid of any one of items to 17, or the composition of item 20, for use in the treatment of a galectin-1-expressing cancer in a subject.

43. The monovalent antibody or composition for use according to item 37, wherein the cancer is of epithelial origin.

44. The monovalent antibody, nucleic acid or composition for use according to item 42 or 43, wherein the cancer is a breast cancer, a melanoma, an ovarian cancer or a lymphoma.

45. The monovalent antibody, nucleic acid or composition for use according to any one of items 42 to 44, wherein said monovalent antibody, nucleic acid or composition is for use in combination with a second anti-tumoral agent.

46. The monovalent antibody of any one of items 1 to 14, the nucleic acid of any one of items to 17, or the composition of item 20, for use in the treatment of a disease or condition associated with pathological neovascularization or angiogenesis.

47. The monovalent antibody of any one of items 1 to 14, the nucleic acid of any one of items to 17, or the composition of item 20, for use in the inhibition of tissue or organ fibrosis.

48. The monovalent antibody, nucleic acid or composition for use according to any one of items 42 to 47, wherein the subject is a human subject.

49. Use of the monovalent antibody of any one of items 1 to 14, or the composition of item 20, for the manufacture of a medicament for binding human galectin-1 (hGAL-1).

50. The use according to item 49, wherein said hGAL-1 is expressed at the surface of a cell.

51. Use of the monovalent antibody of any one of items 1 to 14, or the composition of item 20, for the manufacture of a medicament for inhibiting galectin-1-mediated apoptosis in a cell.

52. The use according to item 51, wherein said cell is an immune cell.

53. The use according to item 52, wherein said immune cell is a T lymphocyte.

54. Use of the monovalent antibody of any one of items 1 to 14, the nucleic acid of any one of items 15 to 17, or the composition of item 20, for the manufacture of a medicament for inhibiting the activity of human galectin-1 (hGAL-1).

55. The use according to item 54, wherein said hGAL-1 is expressed at the surface of a cell.

56. Use of the monovalent antibody of any one of items 1 to 14, the nucleic acid of any one of items 15 to 17, or the composition of item 20, for the manufacture of a medicament for the treatment of a galectin-1-expressing cancer in a subject.

57. The use according to item 56, wherein the cancer is of epithelial origin.

58. The use according to item 56 or 57, wherein the cancer is a breast cancer, a melanoma, an ovarian cancer or a lymphoma.

59. The use according to any one of items 56 to 58, wherein said medicament is for use in combination with a second anti-tumoral agent.

60. Use of the monovalent antibody of any one of items 1 to 14, or the composition of item 20, for the manufacture of a medicament for treating a disease or condition associated with pathological neovascularization or angiogenesis.

61. Use of the monovalent antibody of any one of items 1 to 14, the nucleic acid of any one of items 15 to 17, or the composition of item 20, for the manufacture of a medicament for inhibiting tissue or organ fibrosis.

62. The use according to any one of items 56 to 61, wherein the subject is a human subject.

63. A method for detecting a human galectin-1-expressing cell comprising contacting said cell with the monovalent antibody of any one of items 1 to 14.

64. The method of item 63, wherein said monovalent antibody is conjugated to a detectable label.

65. The method of item 64, wherein said detectable label is a fluorescent molecule or a radioisotope.

66. The method of any one of items 63 to 65, wherein said cell is a tumor cell.

67. The method of item 66, wherein said method is for diagnosing and/or monitoring the progression of a galectin-1-positive cancer in a subject.

68. Use of the monovalent antibody of any one of items 1 to 14 for detecting a human galectin-1-expressing cell.

69. The use of item 68, wherein said monovalent antibody is conjugated to a detectable label.

70. The use of item 69, wherein said detectable label is a fluorescent molecule or a radioisotope.

71. The use of any one of items 68 to 70, wherein said cell is a tumor cell.

72. The use of item 71, wherein said use is for diagnosing and/or monitoring the progression of a galectin-1-positive cancer in a subject.

Other objects, advantages and features of the present invention will become more apparent upon reading of the following non-restrictive description of specific embodiments thereof, given by way of example only with reference to the accompanying drawings.

### BRIEF DESCRIPTION OF DRAWINGS

### In the Appended Drawings:

FIG. 1 shows the structure of galectins. Upper panel, Galectins are a family of glycan binding lectins that recognize carbohydrates by conserved carbohydrate-recognition domains (CRDs). The galectins that have been identified in mammals are widely distributed and have multiple physiological roles in normal and disease conditions. They are classified on the basis of their structure into three groups: prototypical galectins that contain one CRD (Galectin-1, 2, 5, 7, 10, 11, 13, 14, 15 and 16); Galectin-3, a chimeric galectin which consists of one CRD covalently linked to tandem repeats of proline- and glycine-rich short domains; and tandem repeat galectins that contain two covalently linked CRDs connected by a small peptide domain of up to 70 amino acids (Galectin-4, 6, 8, 9 and 12). Lower panel, in the classical model, extracellular galectins interact with and stabilize cell-surface glycoproteins. Crosslinking of the receptors stabilizes the expression of these receptors and triggers a cascade of transmembrane signaling events associated with apoptosis or cellular activation.

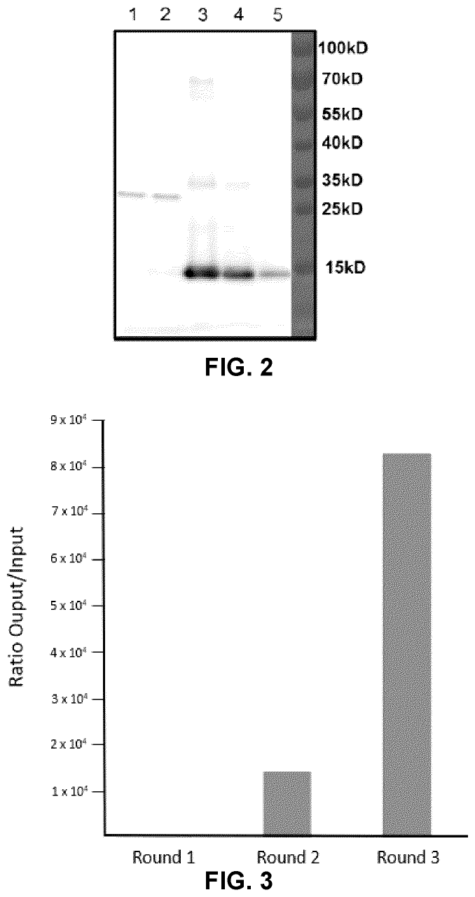

FIG. 2 is a Western blot using a Streptavidin-HRP conjugate (Thermo Fischer) showing successful biotinylation of human GAL-1 that was used for selection of galectin-1-specific nanobodies by phage display. Lane 1: GST-Biotin without Streptavidin Magnetic Beads (Dynabeads® M-280 Streptavidin, Life Technologies); Lane 2: GST-Biotin on beads that was used for in an initial round of Phage Display to deplete the library from unspecific binders; Lane 3: Biotinylated GAL-1 without beads; Lanes 4 and 5: Biotinylated galectin-1 on beads for round 1 and rounds 2-3 of Phage display selection, respectively.

FIG. 3 shows the enrichment over three consecutive rounds of Phage Display selection by the ratio Output/Input. Selection was carried out using a phage display library containing 3×10⁹ camelid VHH sequences. For selection, the phage library was first incubated with GST-Biotin beads to remove unspecific binders. Unbound VHHs were then incubated with GAL-1-Biotin beads. A total of three rounds of Phage Display were performed. The depletion step was repeated before each Phage Display round to remove non-specific VHHs.

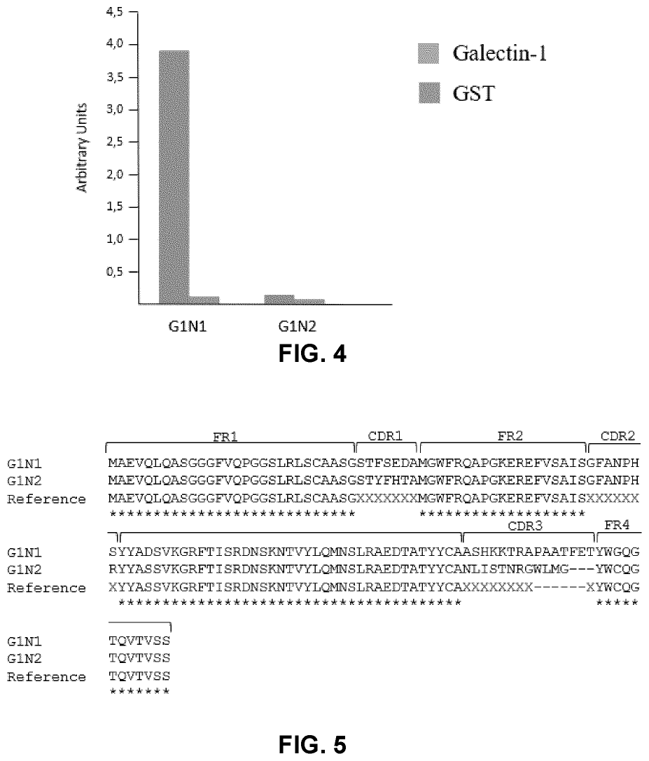

FIG. 4 shows the binding of the 2 positive clones that were identified following the testing of 282 clones that were tested in a 384-well plate ELISA with horseradish peroxidase (HRP)-conjugated anti-M13 antibody (GE Healthcare) and a colorimetric substrate (TMB, tetramethylbenzidine, Thermo Fischer). Both clones showed significant binding in an ELISA test in the presence of GAL-1-Biotin and very low signal in the presence GST-Biotin. The G1N1 clone was considered a strong positive hit while the G1N2 showed only weak binding.

FIG. 5 shows the amino acid sequence of the G1N1 (SEQ ID NO:8) and G1N2 (SEQ ID NO:9) nanobodies (Nbs). Reference is a nanobody with the complementary-determining regions (CDRs) replaced by “X”. The residues forming the CDRs and FRs have been assigned according to the nomenclature set forth in Moutel et al., eLife 2016; 5:e162.

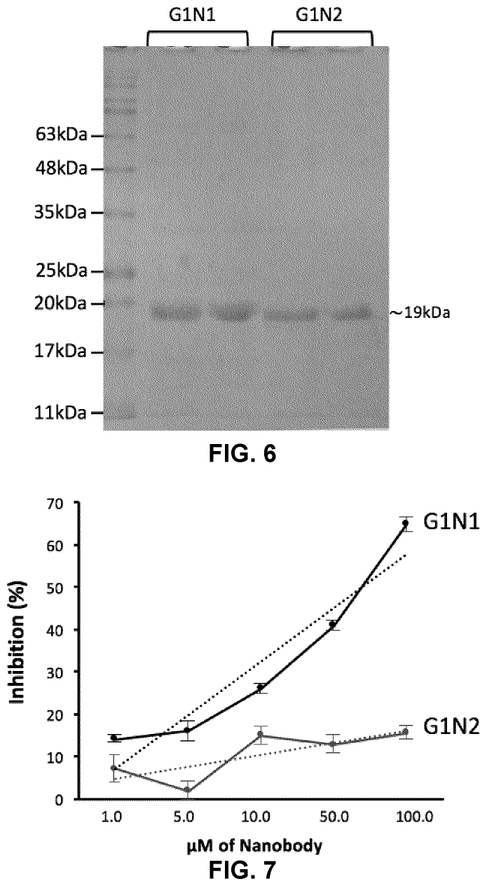

FIG. 6 shows the production and purification of GAL-1-specific Nbs. DNA sequences encoding GAL-1-specific Nbs were inserted into the basic pHEN2 vector for production in E. coli. The SDS-PAGE analysis shows purification of GAL-1-specific Nb #1 (G1N1) and Nb #2 (G1N2) by standard metal (Ni)-affinity chromatography.

FIG. 7 shows the ability of G1N1 and G1N2 to inhibit galectin-1-induced apoptosis of human T cells. Briefly, Jurkat T cells were incubated with recombinant human GAL-1 (rhGAL-1; 2.25 μM) for 4h at 37° C. in presence of increasing concentrations of G1N1 and G1N2. Apoptosis was measured by flow cytometry using conventional annexin V/propidium iodide staining. Results represent data recorded on 5000 cells. Errors bars represent standard deviation.

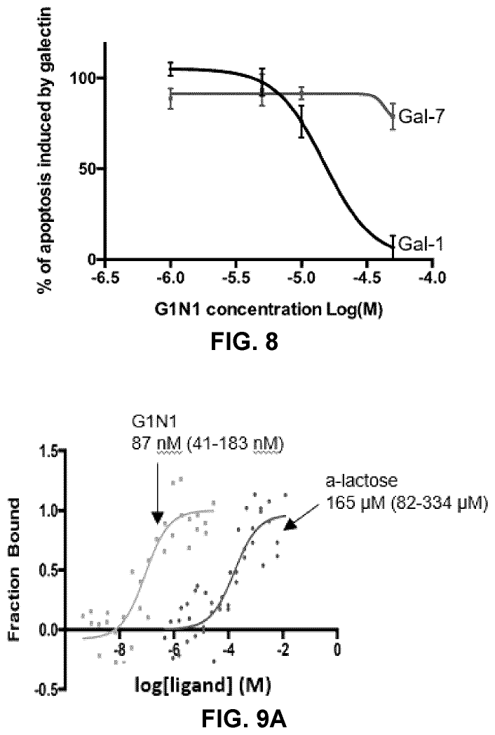

FIG. 8 shows the ability of G1N1 to inhibit GAL-1-, but not GAL-7-induced apoptosis of human T cells. Jurkat T cells were incubated with increasing concentrations of G1N1 pre-incubated with rhGAL-1 (2.25 μM) for 4 h at 37° C., after which apoptosis was measured by flow cytometry using Annexin V/propidium iodide staining. 5000 events were recorded. Errors bars represent standard deviation. No significant inhibition was detected using human GAL-7 (10 μM).

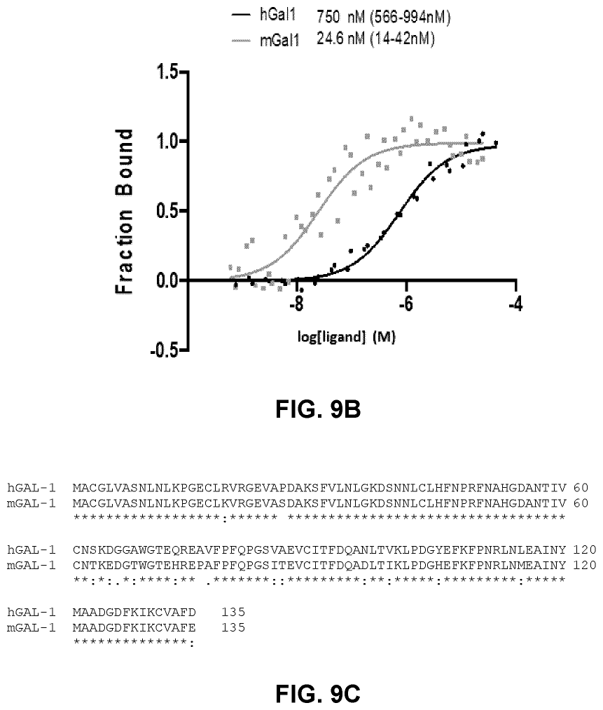

FIGS. 9A-C show the binding affinities evaluated by microscale thermophoresis (MST) using various ligands over a range of 16 ligand concentrations allowing to obtain a full concentration-response curve. FIG. 9A: Red-NHS labeled human GAL-1 (5 nM) incubated with G1N1 or alpha-lactose (a-lactose). FIG. 9B: Red-NHS labeled G1N1 (2 nM) was used to study the affinity of G1N1 for mouse and human galectin-1. In the same experiment, no binding was detected at 10 μM for human GAL-7, mouse GAL-7, human GAL-8 and human GAL-13. All experiments were performed in 20 mM Tris-HCl 150 mM NaCl and 0.1% F127. Binding affinities (Kd values) were determined from the fitted curves (three parameter dose-response curve) obtained from data of at least 3 independent experiments. For each value, 95% confidence intervals (Cl) are indicated. FIG. 9C shows an amino acid sequence alignment of human galectin-(hGAL-1, SEQ ID NO:10) and mouse galectin-1 (mGAL-1, SEQ ID NO:15).

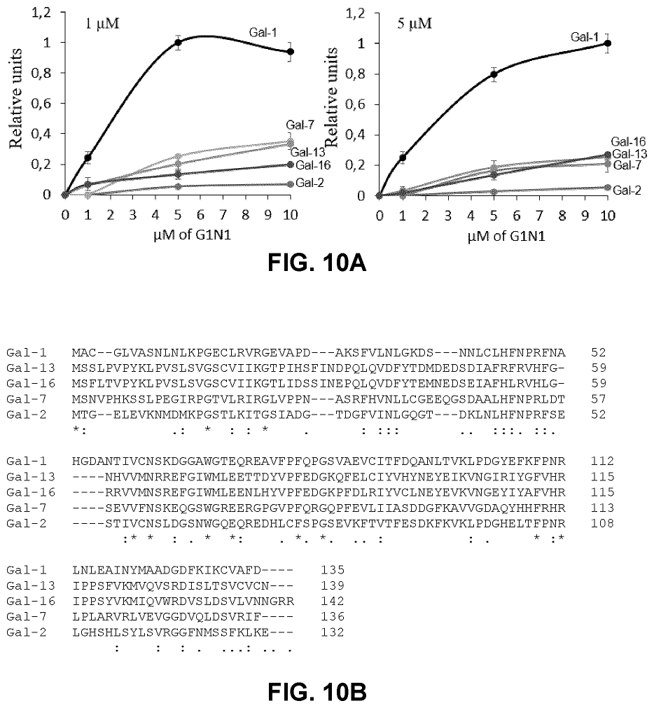

FIG. 10A shows the binding of G1N1 to human galectins as measured by ELISA. Briefly, human galectins were immobilized at 1 and 5 μM for 16 h at 4° C. After a blocking step with PBS containing 10% (v/v) BSA (blocking buffer), increasing concentrations of G1N1 were added to each well and incubated for 1 h at room T° C. Binding of G1N1 was revealed using a goat anti-His-tag polyclonal ( 1/1000) antibody (Bio-Rad, ON, Canada) and a donkey anti-goat IgG ( 1/5000) conjugated to horseradish peroxidase (HRP) (R&D Systems, MN, USA). The colorimetric assay was carried out with 3,3′,5,5′-tetramethylbenzidine (Sigma-Aldrich, ON, Canada) according to the manufacturers recommendations.

FIG. 10B shows an amino acid sequence alignment of human galectin-1 (SEQ ID NO:10), galectin-13 (SEQ ID NO:11), galectin-16 (SEQ ID NO:12), galectin-7 (SEQ ID NO:13) and galectin-2 (SEQ ID NO:14).

### DETAILED DISCLOSURE

The use of the terms “a” and “an” and “the” and similar referents in the context of describing the invention are to be construed to cover both the singular and the plural, unless otherwise indicated herein or clearly contradicted by context.

The terms “comprising”, “having”, “including”, and “containing” are to be construed as open-ended terms (i.e., meaning “including, but not limited to”) unless otherwise noted.

Recitation of ranges of values herein are merely intended to serve as a shorthand method of referring individually to each separate value falling within the range, unless otherwise indicated herein, and each separate value is incorporated into the specification as if it were individually recited herein. All subsets of values within the ranges are also incorporated into the specification as if they were individually recited herein.

Similarly, herein a general chemical structure with various substituents and various radicals enumerated for these substituents is intended to serve as a shorthand method of referring individually to each and every molecule obtained by the combination of any of the radicals for any of the substituents. Each individual molecule is incorporated into the specification as if it were individually recited herein. Further, all subsets of molecules within the general chemical structures are also incorporated into the specification as if they were individually recited herein.

All methods described herein can be performed in any suitable order unless otherwise indicated herein or otherwise clearly contradicted by context.

The use of any and all examples, or exemplary language (“e.g.”, “such as”, etc.) provided herein, is intended merely to better illustrate the invention and does not pose a limitation on the scope of the invention unless otherwise claimed.

Herein, the term “about” has its ordinary meaning. The term “about” is used to indicate that a value includes an inherent variation of error for the device or the method being employed to determine the value, or encompass values close to the recited values, for example within 10% or 5% of the recited values (or range of values).

Unless otherwise defined, all technical and scientific terms used herein have the same meaning as commonly understood by one of ordinary skill in the art to which this invention belongs.

Any and all combinations and subcombinations of the embodiments and features disclosed herein are encompassed by the present disclosure.

In the studies described herein, the present inventors have developed cameloid antibodies (nanobodies, Nbs, also called single domain V_{HH} antibodies), which are particularly well-suited for dimer-interference and blocking the GBS of galectins given their small size. First, Nbs bind antigenic epitopes by virtue of a single (monovalent) and variable domain encoded in the heavy chain fragment [Hamers-Casterman et al., 2013]. This is an important issue in the case of galectin because in contrast to conventional (multivalent) antibodies (Abs), binding of monovalent Nbs on galectin-bound cell surface glycoreceptors cannot trigger intracellular signals induced by cross-linking of glycoreceptors. Secondly, Nbs can recognize epitopes that may be inaccessible to conventional Abs because of their extended convex-shaped paratope. This is possible because the hypervariable region of Nbs is made of a single stretch of amino acids (a.a.) composed of flexible peptide loops, including a relatively long complementary determining region (CDR)-3 loop that is extended and made of 17 a.a on average (compared to 12 a.a. in humans). This confers Nbs with a unique antigen-binding mode capable of targeting hidden (poorly immunogenic) epitopes. The structure of their antigen-binding region is thus ideally suited for targeting epitopes that are not accessible by conventional antibodies, which harbor relatively flat paratopes. Moreover, in contrast to conventional (multivalent) antibodies, binding of monovalent Nbs on galectin-bound cell surface glycoreceptors cannot trigger intracellular signals induced by cross-linking of glycoreceptors. Finally, another important consideration is that Nbs usually exhibit high affinity for their ligands, often in the subnanomolar range. All of these features suggest that Nbs are ideally suited for inhibiting galectins such as galectin-1 (GAL-1). In the studies disclosed in the present disclosure, the generation of a specific and high affinity camelid antibody against human galectin-1 is reported. This nanobody was shown to be highly specific for human galectin-1 to effectively neutralize the ability of galectin-1 to kill human T cells.

Accordingly, in a first aspect, the present disclosure provides a monovalent antibody that specifically binds to human GAL-1. In an embodiment, the present disclosure provides a monovalent antibody that specifically binds to the dimer interface of hGAL-1. In an embodiment, the present disclosure provides a monovalent antibody that specifically binds to the glycan-binding site (GBS) of hGAL-1. In an embodiment, the monovalent antibody inhibits or interferes with human GAL-1 homodimerization. In another embodiment, the monovalent antibody inhibits or interferes with human GAL-1 activity, for example GAL-1-induced killing of cells such as human activated T cells.

The term “monovalent antibody” refers to an antibody that comprises a single monomeric variable antibody domain, and thus a single set of complementary determining regions (CDRs).

Examples of monovalent antibodies include single-domain antibodies (sdAbs, also called nanobodies), camelid antibodies (e.g., from dromedaries, camels, llamas, alpacas), V_{H}H fragments and V_{NAR} fragments. In an embodiment, the monovalent antibody is a nanobody. Single domain antibodies may be derived from any species including mouse, human, camel, llama, goat, rabbit, and bovine. For example, naturally occurring V_{H}H molecules can be derived from antibodies raised in Camelidae species, for example in camel, dromedary, alpaca and guanaco. Synthetic V_{H}H molecules may also be identified/generated using library of humanized nanobodies (see, e.g., Moutel et al., eLife 2016; 5:e16228; Salema et al., MAbs vol. 8, No. 7, 1286-1301; Gene, R W et al., (2015) J Immunol Methods 416, 29-39; Kumaran, J. (2012). Methods Mol Biol 911, 105-124).

GAL-1 is composed of two subunits of 14.5 kDa (135 a.a.) present in a dynamic dimerization equilibrium. It recognizes multiple galactose-βi-4-N-acetyl-glucosamine (N-acetyl-lactosamine [LacNAc]) units present on the branches of N- or O-linked glycans on diverse cell surface receptors, including CD45, CD43, CD69, pre-BCR, and vascular endothelial growth factor R2. Within the immune system, Gal-1 is synthesized and secreted by a wide range of cells, including activated T and B cells, macrophages, Foxp3⁻regulatory T cells (Tregs), tolerogenic dendritic cells (DCs), γō T cells, microglia, and myeloid-derived suppressor cells. Remarkably, GAL-1 expression is prominent in immune-privileged sites, such as placenta, testis, and the eye, and is significantly up- or downmodulated in inflammatory conditions, including microbial infection, autoimmunity, allergy, cancer, reproductive disorders, neurodegenerative diseases, and myocardial infarction. GAL-1 has been shown to control T cell survival by interacting with different components of the cell death machinery. Through binding to N- and O-glycans present in CD45, CD43, and CD7, or by sensitizing T cells to the Fas-mediated pathway, exogenous GAL-1 has been shown to engage cell death programs. GAL-1 has been shown to be involved in tumor-immune escape in several tumor models including lung carcinoma, breast carcinoma, pancreatic carcinoma, ovarian carcinoma, glioblastoma, neuroblastoma, Hodgkin lymphoma and T cell lymphoma. Also, the amount of extracellular of galectin-1 is altered in a variety of cancer cell types including melanoma, ovarian, lung, prostate, bladder, thyroid, pancreatic, head-neck, cervical, uterine, and colorectal cancers. In addition, galectin-1 is often overexpressed in the stroma surrounding tumor cells (Cousin and Cloninger, Int J Mol Sci. 2016 September; 17(9): 1566; Sundblad et al., J Immunol Dec. 1, 2017, 199 (11) 3721-3730).

In an embodiment, the monovalent antibody disclosed herein may be used for the treatment of any of the diseases/cancers defined above.

In an embodiment, the monovalent antibody comprises the following combination of complementarity determining regions (CDRs): a CDR1 comprising or consisting of an amino acid sequence having at least 80%, 85% or 90% identity with the sequence STFSEDA (SEQ ID NO:1); a CDR2 comprising or consisting of an amino acid sequence having at least 80%, 85% or 90% identity with the sequence GFANPHS (SEQ ID NO:2); and a CDR3 comprising or consisting of an amino acid sequence having at least 80%, 85% or 90% identity with the sequence ASHKKTRAPAATFET (SEQ ID NO:3).

Although it will be appreciated that the skilled person will be able to provide for various single-domain antibodies based on the various CDR1, CDR2, CDR3 as disclosed herein, as well as the other sequences provided (including the various framework sequences and the full-length sequence of the single-domain antibodies), preferably the single-domain antibody has a CDR1, CDR2 and a CDR3 as shown in FIG. 5, and conservative sequence variants thereof. In other words, a single-domain antibody according to the present disclosure preferably comprises the CDR1 and the CDR2 and the CDR3 shown in FIG. 5. As will be appreciated by the skilled person, also included are conservative sequence variants of the CDR1, CDR2 and CDR3 combinations as disclosed in FIG. 5. These amino acid changes can typically be made without altering the biological activity, function, or other desired property of the antibody, such as its affinity or its specificity for antigen. In general, single amino acid substitutions in nonessential regions of an antibody do not substantially alter biological activity. Furthermore, substitutions of amino acids that are similar in structure or function are less likely to disrupt the antibody's biological activity. One or more of the CDRs may be mutated to increase the affinity and/or specificity of the monovalent antibody for hGAL-1, e.g., to generate an affinity-matured monovalent antibody.

In an embodiment, one or two residues in the above-noted CDRs sequences are substituted. In a further embodiment, one residue in the above-noted CDRs sequences is substituted.

In an embodiment, the monovalent antibody comprises the following combination of complementarity determining regions (CDRs): a CDR1 comprising or consisting of the sequence STFSEDA (SEQ ID NO:1); a CDR2 comprising or consisting of the sequence GFANPHS (SEQ ID NO:2); and a CDR3 comprising or consisting of the sequence ASHKKTRAPAATFET (SEQ ID NO:3).

In an embodiment, the monovalent antibody comprises: (i) a framework region (FR) 1 comprising or consisting of an amino acid sequence having at least 50%, 60%, 70%, 75%, 80%, 85%, 90% or 95% identity with the sequence MAEVQLQASGGGFVQPGGSLRLSCAASG (SEQ ID NO:4); (ii) a FR2 comprising or consisting of an amino acid sequence having at least 50%, 60%, 70%, 75%, 80%, 85%, 90% or 95% identity with the sequence MGWFRQAPGKEREFVSAIS (SEQ ID NO:5); (iii) a FR3 comprising or consisting of an amino acid sequence having at least 50%, 60%, 70%, 75%, 80%, 85%, 90% or 95% identity with the sequence YYADSVKGRFTISRDNSKNTVYLQMNSLRAEDTATYYCA (SEQ ID NO:6); (iv) a FR4 comprising or consisting of an amino acid sequence having at least 50%, 60%, 70%, 75%, 80%, 85%, 90% or 95% identity with the sequence YWGQGTQVTVSS (SEQ ID NO:7); or (v) any combination of (i) to (iv).

In an embodiment, the monovalent antibody comprises: (i) a framework region (FR) 1 comprising or consisting of the sequence MAEVQLQASGGGFVQPGGSLRLSCAASG (SEQ ID NO:4); (ii) a FR2 comprising or consisting of the sequence MGWFRQAPGKEREFVSAIS (SEQ ID NO:5); (iii) a FR3 comprising or consisting of the sequence YYADSVKGRFTISRDNSKNTVYLQMNSLRAEDTATYYCA (SEQ ID NO:6); (iv) a FR4 comprising or consisting of the sequence YWGQGTQVTVSS (SEQ ID NO:7); or (v) any combination of (i) to (iv).

In an embodiment, the monovalent antibody comprises or consists of an amino acid sequence having at least 80%, 85%, 90%, 95%, 96%, 97%, 98% or 99% identity with the sequence of the antibody G1N1 depicted at FIG. 5. In a further embodiment, the monovalent antibody comprises or consists of the sequence of the antibody G1N1 depicted at FIG. 5.

Variations in the monovalent antibody described herein, can be made, for example, using any of the techniques and guidelines for conservative and non-conservative mutations set forth, for instance, in U.S. Pat. No. 5,364,934. Variations may be a substitution, deletion or insertion of one or more codons encoding the monovalent antibody that results in a change in the amino acid sequence as compared with the native sequence antibody. Optionally the variation is by substitution of at least one amino acid with any other amino acid (including naturally occurring amino acids as well as amino acid analogs) in one or more of the domains of the monovalent antibody. Amino acid substitutions can be the result of replacing one amino acid with another amino acid having similar structural and/or chemical properties, such as the replacement of a leucine with a serine, i.e., conservative amino acid replacements. Insertions or deletions may optionally be in the range of about 1 to 5 amino acids. The variation allowed may be determined by systematically making insertions, deletions or substitutions of amino acids in the sequence and testing the resulting monovalent antibody variants for activity exhibited by the “native” (or reference) monovalent antibody.

“Identity” refers to sequence identity between two polypeptides. Identity can be determined by comparing each position in the aligned sequences. Methods of determining percent identity are known in the art, and several tools and programs are available to align amino acid sequences and determine a percentage of identity including EMBOSS Needle, ClustalW, SIM, DIALIGN, etc. As used herein, a given percentage of identity with respect to a specified subject sequence, or a specified portion thereof, may be defined as the percentage of amino acids in the candidate derivative sequence identical with the amino acids in the subject sequence (or specified portion thereof), after aligning the sequences and introducing gaps, if necessary to achieve the maximum percent sequence identity, as generated by the Smith Waterman algorithm (Smith & Waterman, J. Mol. Biol. 147 147: 195-7 (1981)) using the BLOSUM substitution matrices (Henikoff & Henikoff, Proc. Natl. Aced. Sci. USA 89:10915-9 (1992)) as similarity measures. A“% identity value” is determined by the number of matching identical amino acids divided by the sequence length for which the percent identity is being reported.

In an embodiment, the monovalent antibodies of the present disclosure may be subjected to in vitro affinity maturation. A library comprising variants of the monovalent antibodies disclosed herein may be generated and screened to identity monovalent antibodies having improved affinity and/or specificity for the target antigen (hGAL-1). Thus, in another aspect, the present disclosure provides a method for identifying affinity-matured monovalent antibodies specific for hGAL-1 comprising: (i) generating a library of test monovalent antibodies, wherein said test monovalent antibodies comprises one or more mutations (point mutations, substitutions) relative to the parent monovalent antibody disclosed herein (FIG. 5); (ii) selecting the monovalent antibodies that binds to hGAL-1 with higher affinity than the parent monovalent antibody, thereby identifying affinity-matured monovalent antibodies. In an embodiment, the one or more mutations is in one or more of the CDRs disclosed herein. In an embodiment, the test monovalent antibody comprises 15 mutations or less relative to the parent monovalent antibody. In an embodiment, the test monovalent antibody comprises 10 mutations or less relative to the parent monovalent antibody. In embodiments, the test monovalent antibody comprises 9, 8, 7, 6, or 5 mutations or less relative to the parent monovalent antibody. In an embodiment, the affinity of the affinity-matured monovalent antibody for hGAL-1 is at least 2-fold that of the parent monovalent antibody. In embodiments, the affinity of the affinity-matured monovalent antibody for hGAL-1 is at least 5-, 10-, 20-, 50- or 100-fold that of the parent monovalent antibody.

Modifications to the C or N-terminal V_{H} framework sequence may be made to the monovalent antibodies of the disclosure to improve their properties. For example, the V_{H} domain may comprise C or N-terminal extensions or deletions. C-terminal extensions can be added to the C terminal end of a V_{H} domain.

In one embodiment, the monovalent antibodies of the disclosure comprise C-terminal extensions or deletions of from 1 to 50, or more residues, for example 1 to 25, e.g., 1, 2, 3, 4, 5, 6, 7, 8, 9 or 10 additional amino acids.

Additional C or N-terminal residues can be linkers that are used to conjugate the monovalent antibodies of the disclosure to another moiety, or tags that facilitate the detection of the molecule (hGAL-1). Such tags are well known in the art and include for example polyhistidine tags (His-tags), polyarginine tags, polyaspartate tags, polycysteine tags, polyphenylalanine tags, glutathione S-transferase (GST) tags, Maltose binding protein (MBP) tags, calmodulin binding peptide (CBP) tags, Streptavidin/Biotin-based tags, HaloTag®, Profinity eXact® tags, epitope tags (such as FLAG, hemagglutinin (HA), HSV, S/S1, c-myc, KT3, T7, V5, E2, and Glu-Glu epitope tags), reporter tags such as s-galactosidase (s-gal), alkaline phosphatase (AP), chloramphenicol acetyl transferase (CAT), and horseradish peroxidase (HRP) tags (see, e.g., Kimple et al., Cuff Protoc Protein Sci. 2013; 73: Unit-9.9).

The monovalent antibody according to the present disclosure may comprise at least one constant domain, e.g., a constant domain of a light and/or heavy chain, or a fragment thereof. For example, the monovalent antibody may comprise a Fragment crystallizable (Fc) region or domain of the constant heavy chain of an antibody. The Fc fragment may comprise two or three constant domains, e.g., a CH₂ domain and CH₃ domain. The Fc region may be obtained from a human IgG1, a human IgG4, or a variant of a human IgG1 or IgG4 having up to ten amino acid modifications, for example. In an embodiment, the Fc fragment comprises or consists of the CH₂ domain and CH₃ domain of a human antibody, preferably a human IgG such as IgG1. The presence of an Fc domain on the monovalent antibody may promote antibody-mediated activities such as complement-dependent cytotoxicity (CDC), antibody-dependent cellular cytotoxicity (ADCC) and/or antibody-dependent cellular phagocytosis (ADCP), i.e., the cytotoxic killing or phagocytosis of cells bound by the monovalent antibody (e.g., GAL-1-expressing tumor cells).

The monovalent antibody according to the present disclosure may be linked to other function or non-functional groups, for example the monovalent antibody may be conjugated to a label (e.g., a biotin label, a fluorescent label, an enzyme label, a coenzyme label, a chemiluminescent label), a nanoparticle, a drug (e.g., a chemotherapeutic agent, an anti-inflammatory drug), a peptide, a nucleic acid, a toxin, an enzyme, a radioisotope, a half-life extending moiety (e.g., PEGylation, using a serum albumin protein), a therapeutic molecule or any other chemical moiety. The monovalent antibody may be used to target hGAL-1 expressing cells (e.g., cancer cells), or may be used to detect hGAL-1 and/or cells expressing hGAL-1, in diagnostic, prognostic, disease monitoring and medical imaging applications.

In an embodiment, the monovalent antibody according to the present disclosure is conjugated to one or more therapeutic or active agents (e.g., a drug), and thus may also be used therapeutically to deliver the therapeutic agent(s) (e.g., anti-tumor agent or any other agent useful for the treatment of the disease or condition or for relieving one or more symptoms) into a cell or tissue, such as a tumor. Any method known in the art for conjugating the monovalent antibody thereof to another moiety (e.g., detectable moiety, active agent) may be employed (Hermanson, Bioconjugate Techniques, 3^{rd} edition, 2013, Academic Press, Inc., San Diego).

Nanobodies may be produced in various expression systems including E. coli, yeasts, or filamentous fungi (see, for example, Harmsen and De Haard, Appl Microbiol Biotechnol. 2007 November; 77(1): 13-22).

A further aspect of the present disclosure provides nucleic acids encoding the monovalent antibody according to the present disclosure. The isolated nucleic acid may be a synthetic DNA, a non-naturally occurring mRNA, or a cDNA, for example. The nucleic acid may be inserted within a plasmid, vector, or transcription or expression cassette. The nucleic acids encoding the monovalent antibody according to the present disclosure may be made and the expressed monovalent antibodies may be tested using conventional techniques well known in the art. In some embodiments, the nucleic acid encoding the monovalent antibody described herein can be maintained in the vector in a host cell. In some embodiments, the nucleic acid is an expression vector. In some embodiments, the nucleic acid sequence encoding the monovalent antibody can be maintained in the vector in a host cell. In embodiment, the nucleic acid(s) (DNA, mRNA) encoding the monovalent antibody described herein of the disclosure is comprised within a vesicle such as lipid nanoparticles (e.g., liposomes) or any other suitable vehicle. In an embodiment, the nucleic acid is an mRNA and is encapsulated into nanoparticulate delivery vehicles (see, e.g., Van Hoecke and Roose (2019) How mRNA therapeutics are entering the monoclonal antibody field, J. Transl. Med. 17, 54. https://doi.org/10.1186/s12967-019-1804-8; Sanz and Alvarez-Vallina (2021) Engineered mRNA and the Rise of Next-Generation Antibodies, Antibodies 10(4):37. https://doi.org/10.3390/antib10040037).

In another aspect, the present invention provides a cell, for example a recombinant host cell, comprising the above-noted nucleic acids and expressing the monovalent antibody according to the present disclosure. Methods of preparing monovalent antibodies comprise expressing the encoding nucleic acid(s) in a host cell under conditions to produce the antibodies, and recovering the antibodies. The process of recovering the antibodies may comprise isolation and/or purification of the antibodies. The method of production may comprise formulating the antibodies into a composition including at least one additional component, such as a pharmaceutically acceptable excipient.

The term “recombinant host cell” (or simply “host cell”), as used herein, is intended to refer to a cell into which exogenous DNA has been introduced. It should be understood that such terms are intended to refer not only to the particular subject cell, but, to the progeny of such a cell. Because certain modifications may occur in succeeding generations due to either mutation or environmental influences, such progeny may not, in fact, be identical to the parent cell, but are still included within the scope of the term “host cell” as used herein. Preferably host cells include prokaryotic and eukaryotic cells selected from any of the Kingdoms of life. Preferred eukaryotic cells include protist, fungal, plant and animal cells. Most preferably host cells include but are not limited to the prokaryotic cell line E. Coli; mammalian cell lines CHO, HEK 293 and COS; the insect cell line Sf9; the fungal cell Saccharomyces cerevisiae, plant cells, or algae cells.

In another embodiment, the host cell is an immune cell. The anti-hGAL-1 monovalent antibody described herein may be used as a chimeric antigen receptor (CAR) to produce CAR T cells, CAR NK cells, etc. CAR combines a ligand-binding domain (e.g., antibody or antibody fragment) that provides specificity for a desired antigen (e.g., hGAL-1) with an activating intracellular domain (or signal transducing domain) portion, such as a T cell or NK cell activating domain, providing a primary activation signal. Nanobodies capable of binding to molecules expressed by tumor cells are commonly used as CAR. Thus, in another aspect, the present disclosure provides a host cell, preferably an immune cell such as a T cell or NK cell, expressing the monovalent antibody described herein.

The CAR of the present disclosure may also comprise a transmembrane domain which spans the membrane. The transmembrane domain may be derived from a natural polypeptide, or may be artificially designed. The transmembrane domain derived from a natural polypeptide can be obtained from any membrane-binding or transmembrane protein. For example, a transmembrane domain of a T cell receptor α or β chain, CD28, CD3-epsilon, CD45, CD4, CD5, CD8, CD9, CD16, CD22, CD33, CD37, CD64, CD80, CD86, CD134, CD137, ICOS, CD154, or a GITR can be used. The artificially designed transmembrane domain is a polypeptide mainly comprising hydrophobic residues such as leucine and valine. It is preferable that a triplet of phenylalanine, tryptophan and valine is found at each end of the synthetic transmembrane domain. In preferred embodiments, the transmembrane domain is derived from CD28 or CD8, which give good receptor stability.

Preferred examples of signal transducing domain for use in a CAR can be the cytoplasmic sequences of the T cell receptor and co-receptors that act in concert to initiate signal transduction following antigen receptor engagement, as well as any derivate or variant of these sequences and any synthetic sequence that has the same functional capability. Signal transduction domain comprises two distinct classes of cytoplasmic signaling sequence, those that initiate antigen-dependent primary activation, and those that act in an antigen-independent manner to provide a secondary or co-stimulatory signal. Primary cytoplasmic signaling sequence can comprise signaling motifs which are known as immunoreceptor tyrosine-based activation motifs of ITAMs. ITAMs are well defined signaling motifs found in the intracytoplasmic tail of a variety of receptors that serve as binding sites for Syk/ZAP70 class tyrosine kinases. Examples of ITAM used in the invention can include as non-limiting examples those derived from TCRzeta, FcRgamma, FcRbeta, FcRepsilon, CD3gamma, CD3delta, CD3epsilon, CD5, CD22, CD79a, CD79b and CD66d.

The CAR of the present disclosure may also comprise one or more co-stimulatory domains such as human CD28, 4-1BB (CD137), ICOS-1, CD27, OX40 (CD137), DAP10, and GITR (AITR). In embodiment, the CAR is a third generation and comprises two co-stimulating domains such as CD28 and 4-1BB.

The CAR of the present disclosure may also comprise a signal peptide N-terminal to the anti-GAL-1 monovalent antibody described herein so that when the CAR is expressed inside a cell, such as a T-cell, the nascent protein is directed to the endoplasmic reticulum and subsequently to the cell surface, where it is expressed. The core of the signal peptide may contain a long stretch of hydrophobic amino acids that has a tendency to form a single alpha-helix. The signal peptide may begin with a short positively charged stretch of amino acids, which helps to enforce proper topology of the polypeptide during translocation. At the end of the signal peptide there is typically a stretch of amino acids that is recognized and cleaved by signal peptidase. Signal peptidase may cleave either during or after completion of translocation to generate a free signal peptide and a mature protein. The free signal peptides are then digested by specific proteases. As an example, the signal peptide may derive from human CD8 or GM-CSF, or a variant thereof having 1 or 2 amino acid mutations provided that the signal peptide still functions to cause cell surface expression of the CAR.

The CAR of the present disclosure may comprise a spacer sequence as a hinge to connect the anti-GAL-1 monovalent antibody described herein with the transmembrane domain and spatially separate antigen binding domain from the endodomain. A flexible spacer allows to the binding domain to orient in different directions to enable its binding to the desired antigen (e.g., hGAL-1). The spacer sequence may, for example, comprise an IgG1 Fc region, an IgG1 hinge or a CD8 stalk, or a combination thereof.

The term “vector”, as used herein, is intended to refer to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome.

Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

Introducing such nucleic acids into a host cell can be accomplished using techniques well known in the art. For eukaryotic cells, suitable techniques may include calcium phosphate transfection, DEAE-Dextran, electroporation, liposome-mediated transfection, and transduction using retroviruses or other viruses, for example. For bacterial cells, suitable techniques may include calcium chloride transformation, electroporation, and transfection using bacteriophage. The introduction may be followed by causing or allowing expression from the nucleic acid, e.g. by culturing host cells under conditions for expression of the gene. In one embodiment, the nucleic acid of the invention is integrated into the genome, e.g., chromosome, of the host cell. Integration may be promoted by inclusion of sequences that promote recombination with the genome, in accordance with standard techniques.

Systems for cloning and expression of a polypeptide in a variety of different host cells are well known. Suitable host cells include bacteria, mammalian cells, plant cells, insect cells, fungi, yeast and transgenic plants and animals. Mammalian cell lines available in the art for expression of a heterologous polypeptide include Chinese hamster ovary (CHO) cells, HeLa cells, baby hamster kidney cells, mouse melanoma cells, rat myeloma cells, human embryonic kidney cells, e.g., HEK293 cells, human embryonic retina cells, and many others. The expression of antibodies and antibody fragments in prokaryotic cells, such as E. coli, is well established in the art. For a review, see for example, Pliickthun Bio/Technology 9: 545-551 (1991). Expression in cultured eukaryotic cells is also available to those skilled in the art, as reviewed in Andersen et al. (2002) Curr. Opin. Biotechnol. 13: 117-23, for example.

In another aspect, the present disclosure provides a composition (e.g., a pharmaceutical composition) comprising the above-mentioned monovalent antibody. In an embodiment, the composition further comprises one or more pharmaceutically acceptable carriers, excipient, and/or diluents.

As used herein, “pharmaceutically acceptable” (or “biologically acceptable”) refers to materials characterized by the absence of (or limited) toxic or adverse biological effects in vivo. It refers to those compounds, compositions, and/or dosage forms which are, within the scope of sound medical judgment, suitable for use in contact with the biological fluids and/or tissues and/or organs of a subject (e.g., human, animal) without excessive toxicity, irritation, allergic response, or other problem or complication, commensurate with a reasonable benefit/risk ratio.

The term “pharmaceutically acceptable carriers, excipient, and/or diluents” refers to additives commonly used in the preparation of pharmaceutical compositions and includes, for example, solvents, dispersion media, saline solutions, surfactants, solubilizing agents, lubricants, emulsifiers, coatings, antibacterial and antifungal agents, chelating agents, pH-modifiers, soothing agents, buffers, reducing agents, antioxidants, isotonic agents, absorption delaying agents or the like. Such compositions may be prepared in a manner well known in the pharmaceutical art by mixing the antibody having a suitable degree of purity with one or more optional pharmaceutically acceptable carriers or excipients (see Remington: The Science and Practice of Pharmacy, by Loyd V Allen, Jr, 2012, 22^{nd} edition, Pharmaceutical Press; Handbook of Pharmaceutical Excipients, by Rowe et al., 2012, 7^{th} edition, Pharmaceutical Press). The carrier/excipient can be suitable for administration of the antibody by any conventional administration route, for example, for oral, intravenous, parenteral, subcutaneous, intramuscular, intracranial, intraorbital, ophthalmic, intraventricular, intracapsular, intraspinal, intrathecal, epidural, intracisteral, intraperitoneal, intranasal or pulmonary (e.g., aerosol) administration. In an embodiment, the carrier/excipient is adapted for administration of the antibody by the intravenous or subcutaneous route. In an embodiment, the carriers/excipients are adapted for administration of the antibody by the intravenous route. In another embodiment, the carriers/excipients are adapted for administration of the antibody thereof by the subcutaneous route.

The composition may also comprise one or more additional active agents for the treatment the targeted disease/condition or for the management of one or more symptoms of the targeted disease/condition (e.g., pain killers, anti-nausea agents, etc.), as described in more detail below.

The monovalent antibody of the present disclosure may be used to inhibit any biological, physiological and/or pathological process that involves GAL-1 activity, for example GAL-1 activity associated with dimerization.

In another aspect, the present disclosure provides a method (in vitro or in vivo) for binding to GAL-1, said method comprising contacting said GAL-1 with the monovalent antibody or the composition described herein. In an embodiment, the above-mentioned method is for binding to GAL-1 in a cell or in the extracellular space (since prototypic galectins such as GAL-1 are released by cells via a non-classical secretory pathway). The present disclosure also provides the use of the monovalent antibody or the composition described herein for binding to GAL-1. The present disclosure also provides the use of the monovalent antibody or the composition described herein for the manufacture of a medicament for binding to GAL-1. The method or use for binding to GAL-1 may be useful in diagnostic, disease monitoring, prognostic or therapeutic application, notably to detect GAL-1, to identify and/or target GAL-1-expressing cells (e.g., by CAR cells), to deliver molecules (e.g., cytotoxic agents) to GAL-1-expressing cells.

In another aspect, the present disclosure provides a method (in vitro or in vivo) for inhibiting the activity of GAL-1, said method comprising contacting said GAL-1 with the monovalent antibody or the composition described herein. In an embodiment, the above-mentioned method is for inhibiting the dimerization of GAL-1 in a cell or in the extracellular space (since prototypic galectins such as GAL-1 are released by cells via a non-classical secretory pathway). The present disclosure also provides the use of the monovalent antibody or the composition described herein for inhibiting the activity of GAL-1. The present disclosure also provides the use of the monovalent antibody or the composition described herein for the manufacture of a medicament for inhibiting the activity of GAL-1.

Recombinant hGAL-1 has been shown to kill certain types of cells, such as Jurkat T cells, monocytes and human peripheral T cells, suggesting that GAL-1 has immunosuppressive properties. In another aspect, the present disclosure provides a method for inhibiting GAL-1-mediated apoptosis in a cell, said method comprising contacting said cell with the monovalent antibody or the composition described herein. The present disclosure also provides the use of the monovalent antibody or the composition described herein for inhibiting GAL-1-mediated apoptosis in a cell. The present disclosure also provides the use of the monovalent antibody or the composition described herein for the manufacture of a medicament for inhibiting GAL-1-mediated apoptosis in a cell. In an embodiment, the above-mentioned cell is an immune cell, such as a T lymphocyte or a monocyte. In another aspect, the present disclosure provides a method for inhibiting GAL-1-mediated immunosuppression in a subject, said method comprising administering to said subject an effective amount of the monovalent antibody or the composition described herein. The present disclosure also provides the use of the monovalent antibody or the composition described herein for inhibiting GAL-1-mediated immunosuppression in a subject. The present disclosure also provides the use of the monovalent antibody or the composition described herein for the manufacture of a medicament for inhibiting GAL-1-mediated immunosuppression in a subject. In an embodiment, the subject suffers from a GAL-1-expressing cancer. In an embodiment, the monovalent antibody reduces or inhibits the binding of extracellular GAL-1 to glycoreceptors expressed by infiltrated immune cells.

GAL-1 is expressed in several tissues with a certain preference but not exclusive for cells of mesenchymal origin like fibroblasts and lymphocytes. It is involved in the regulation of cell growth, adhesion, signaling, differentiation, development, immune system and host-pathogen interactions (Blanchard et al., 2016). Expression profiles of galectin-1 in the various stages of cancer progression and its role in the tumor microenvironment have been thoroughly reviewed.

GAL-1 has been found mainly to have an immunosuppressive and anti-inflammatory role (Elola et al., Biochem J. 2015 Jul. 1; 469(1):1-16), although in some cases it may also be proinflammatory. GAL-1 binds specific glycosylation pattern on T-helper cells to selectively induce apoptosis in activated Th1 and Th17 cells. (Perillo et. al., J Natl Cancer Inst 87, 348-353) (Toscano, M. A. et al., Nat Immunol 8: 825-834). The immunosuppressive effect of GAL-1 has suggested that GAL-1 itself, might be a potential treatment for autoimmune and other inflammatory conditions, and this inhibiting its immunosuppressive effect, e.g., in cancer, has also been proposed as a treatment.

GAL-1 has been shown promote angiogenesis under certain circumstances (Hockl et al., GAS. Glyco-nano-oncology: Novel therapeutic opportunities by combining small and sweet.

Treatment of cancer Pharmacol Res. 2016 Feb. 4. pii: S1043-6618(16)00042-6. doi:10.1016/j.phrs.2016.02.005. [Epub ahead of print]) in a way involving its carbohydrate binding-activity. It has been suggested that it might promote tumor angiogenesis by a pathway parallel to VEGF, and thus inhibiting GAL-1 using the monovalent antibody or composition described herein may be anti-angiogenic when inhibition based on anti-VEGF fails. Thus, in another aspect, the present disclosure provides the use of the monovalent antibody or composition described herein for reducing angiogenesis, such as pathological angiogenesis, in a subject. In an embodiment, the pathological angiogenesis is ocular angiogenesis or a disease or condition associated with ocular angiogenesis, e.g., neovascularization related to cancer; and eye diseases, such as age-related macular degeneration, diabetic retinopathy and corneal neovascularization.

There is evidence that GAL-1 may be an endogenous enhancer of TGF-β signaling and myofibroblast activation (Kathiriya et al., Cell Death Discovery 3, 17010-13 (2017)), and thus GAL-inhibition using the monovalent antibody or composition described herein may also be useful in treating fibrosis and adverse tissue remodeling, i.e., for reducing scarring (e.g., aberrant scar formation) and keloid formation.

GAL-1 is frequently over-expressed in low differentiated cancer cells. GAL-1 induces apoptosis in activated T-cells and has a remarkable immunosuppressive effect on autoimmune disease in vivo, and therefore its over-expression in cancers might help the tumor to defend itself against the T-cell response raised by the host. GAL-1-expressing or GAL-1-overexpressing cancers include various carcinomas such as lung carcinoma, breast carcinoma, pancreatic carcinoma, and ovarian carcinoma, glioblastoma, neuroblastoma, Hodgkin lymphoma and T cell lymphoma. Also, the amount of extracellular of GAL-1 is altered in a variety of cancer cell types including melanoma, ovarian, lung, prostate, bladder, thyroid, pancreatic, head-neck, cervical, uterine, and colorectal cancers. Thus, the monovalent antibody or composition comprising same disclosed herein may be used for the treatment of any of the above-noted cancers. In an embodiment, the monovalent antibody or composition reduces or inhibits the binding of GAL-1 to glycosylated residues on cell surface receptors of tumor cells.

In another aspect, the present disclosure provides a method for treating a GAL-1-expressing cancer (e.g., inhibiting tumor growth and/or metastasis) in a subject, said method comprising administering to said subject an effective amount of the monovalent antibody or the composition described herein. The present disclosure also provides the use of the monovalent antibody or the composition described herein for treating a GAL-1-expressing cancer in a subject. The present disclosure also provides the use of the monovalent antibody or the composition described herein for the manufacture of a medicament for treating a GAL-1-expressing cancer in a subject.

In another aspect, the present disclosure provides a method for detecting, diagnosing and/or monitoring the progression of a GAL-1-expressing cancer (e.g., monitoring tumor size and/or metastasis) in a subject, said method comprising administering to said subject an effective amount of the monovalent antibody or the composition described herein. The present disclosure also provides the use of the monovalent antibody or the composition described herein for detecting, diagnosing and/or monitoring the progression of a GAL-1-expressing cancer in a subject. The present disclosure also provides the use of the monovalent antibody or the composition described herein for the manufacture of an agent for detecting, diagnosing and/or monitoring the progression of a GAL-1-expressing cancer in a subject.

In an embodiment, the GAL-1-expressing cancer is of epithelial origin. In another embodiment, the GAL-1-expressing cancer is a breast cancer, a melanoma, an ovarian cancer or a lymphoma. In a further embodiment, the GAL-1-expressing cancer is a breast cancer. In another embodiment, the GAL-1-expressing cancer is an ovarian cancer. In another embodiment, the GAL-1-expressing cancer is a lymphoma. In another embodiment, the cancer is a cancer of neural cells, for example a medulloblastoma, glioblastoma or neuroblastoma.

The amount of the monovalent antibody which is effective for the above-noted activities/therapeutic uses will depend on several factors including the nature and severity of the disease, the chosen prophylactic/therapeutic regimen, the target site of action, the patient's weight, special diets being followed by the patient, concurrent medications being used, the administration route and other factors that will be recognized by those skilled in the art. The dosage will be adapted by the clinician in accordance with conventional factors such as the extent of the disease and different parameters from the patient. Typically, 0.001 to 1000 mg/kg of body weight/day will be administered to the subject. In an embodiment, a daily dose range of about 0.01 mg/kg to about 500 mg/kg, in a further embodiment of about 0.1 mg/kg to about 200 mg/kg, in a further embodiment of about 1 mg/kg to about 100 mg/kg, in a further embodiment of about 10 mg/kg to about 50 mg/kg, may be used. The dose administered to a patient, in the context of the present disclosure should be sufficient to effect/induce a beneficial prophylactic and/or therapeutic response in the patient over time (in the case of a cancer, a decrease in tumor size, inhibition of tumor cell proliferation, increased survival time, etc.). The size of the dose also will be determined by the existence, nature, and extent of any adverse side-effects that accompany the administration. Effective doses may be extrapolated from dose response curves derived from in vitro or animal model test systems. For example, in order to obtain an effective mg/kg dose for humans based on data generated from rat studies, the effective mg/kg dosage in rat may be divided by six.

In an embodiment, the above-mentioned treatment comprises the use/administration of more than one (i.e., a combination of) active/therapeutic agent, including the above-mentioned monovalent antibody. The combination of prophylactic/therapeutic agents and/or compositions of the present disclosure may be administered or co-administered (e.g., consecutively, simultaneously, at different times) in any conventional dosage form. Co-administration in the context of the present disclosure refers to the administration of more than one therapeutic in the course of a coordinated treatment to achieve an improved clinical outcome. Such co-administration may also be coextensive, that is, occurring during overlapping periods of time. For example, a first agent may be administered to a patient before, concomitantly, before and after, or after a second active agent is administered. The agents may in an embodiment be combined/formulated in a single composition and thus administered at the same time. In an embodiment, the one or more active agent(s) is used/administered in combination with one or more agent(s) or treatment currently used to prevent or treat the disorder in question (e.g., agents or treatments currently used in the treatment of cancers, such as radiotherapy, surgery and/or targeted therapy).

In an embodiment, the monovalent antibody described herein is used in combination with one or more chemotherapeutic agents. Examples of chemotherapeutic agents suitable for use in combination with the monovalent antibody described herein include, but are not limited to, vinca alkaloids, agents that disrupt microtubule formation (such as colchicines and its derivatives), anti-angiogenic agents, therapeutic antibodies, EGFR targeting agents, tyrosine kinase targeting agent (such as tyrosine kinase inhibitors), transitional metal complexes, proteasome inhibitors, antimetabolites (such as nucleoside analogs), alkylating agents, platinum-based agents, anthracycline antibiotics, topoisomerase inhibitors, macrolides, retinoids (such as all-trans retinoic acids or a derivatives thereof), geldanamycin or a derivative thereof (such as 17-AAG), immunotherapeutic agents (e.g., immune checkpoint inhibitors such as PD-1/PD-L1 inhibitors and CTLA-4 inhibitors, B7-1/B7-2 inhibitors, CAR T cells) and other cancer therapeutic agents recognized in the art. In some embodiments, chemotherapeutic agents for use in combination with the monovalent antibody described herein comprise one or more of adriamycin, colchicine, cyclophosphamide, actinomycin, bleomycin, duanorubicin, doxorubicin, epirubicin, mitomycin, methotrexate, mitoxantrone, fluorouracil, carboplatin, carmustine (BCNU), methyl-CCNU, cisplatin, etoposide, interferons, camptothecin and derivatives thereof, phenesterine, taxanes and derivatives thereof (e.g., taxol, paclitaxel and derivatives thereof, taxotere and derivatives thereof, and the like), topetecan, vinblastine, vincristine, tamoxifen, piposulfan, nab-5404, nab-5800, nab-5801, Irinotecan, HKP, Ortataxel, gemcitabine, Oxaliplatin, Herceptin®, vinorelbine, Doxil®, capecitabine, Alimta®, Avastin®, Velcade®, Tarceva®, Neulasta®, lapatinib, sorafenib, eriotinib, erbitux, PD-1/PD-L1 inhibitors (e.g., nivolumab, pembrolizumab, atezolizumab), CTLA-4 inhibitors (e.g., Ipilimumab), and derivatives thereof, and the like. In an embodiment, the monovalent antibody or composition comprising same described herein is used in combination with an EGFR or tyrosine kinase targeting agent, for example an EGFR inhibitor (RTK inhibitor). The monovalent antibody or composition comprising same described herein may also be used in combination with one or more additional therapeutic antibodies or antibody fragments, e.g., therapeutic antibodies or antibody fragments used for the treatment of tumors or of one or more of the diseases associated with the activity of GAL-1 described above.

As used herein, the term “subject” is taken to mean warm blooded animals such as mammals, for example, cats, dogs, mice, guinea pigs, horses, bovine cows, sheep and humans. In an embodiment, the subject is a mammal, and more particularly a human.

### MODE(S) FOR CARRYING OUT THE INVENTION

The present invention is illustrated in further details by the following non-limiting examples.

### Example 1: Materials and Methods

Cell line and reagents. The Jurkat cell line was maintained in RPMI 1640 medium. The culture medium was supplemented with 10% [v/v] fetal bovine serum, 2 mmol/L L-glutamine, 10 mM HEPES buffer, and 1 mM sodium pyruvate. All cell culture products were purchased from Life Technologies® (Burlington, ON, Canada).

Production and purification of recombinant galectins. Codon-optimized cDNA were synthesized and subcloned into the pET-22b(+) vector for production in E. coli BL21 (DE3) cells as previously described [Vladoiu et al., 2015]. Purification of galectin-1, -2, -7 and -13 was carried out using conventional lactose affinity and/or asialofetuin (ASF) chromatography. Each preparation was dialyzed against phosphate-buffered saline (PBS) to remove residual lactose. Galectin-16 was carried out from inclusion bodies using a 10% sodium docecyl sulfate (SDS) solution and refolded in 25 mM Tris, 10% glycerol, 1M 2-methyl-2,4-pentanediol for 24 h at room temperature.

Generation and production of single chain camelid antibodies. Galectin-specific single chain (VHH) camelid antibodies were obtained following in vitro screening of a non-immune humanized synthetic single domain antibodies (hsd2Ab) library that contains more than 3×10⁹ clones expressed at the surface of M13 phage [Moutel et al., 2016]. The panning was carried out against immobilized recombinant human galectin-1 devoid of carbohydrate in its glycan binding site (GBS). Prior to the phage display selection, galectin-1-biotin and GST-Biotin were bound to streptavidin magnetic beads (Dynabeads® M-280 Streptavidin, Life Technologies) with a 50 nM final concentration of biotinylated protein for the first round and 10 nM final concentration of biotinylated protein for the second and third rounds. The successful binding of the biotinylated proteins on the streptavidin beads was controlled by SDS-polyacrylamide gel electrophoresis/Western blot using a streptavidin-horseradish peroxydase (HRP) conjugate (Thermo Fischer) (FIG. 2). Following three-rounds of phage display, clones were picked randomly and analyzed by non-adsorbed phage ELISA using HRP-conjugated anti-M13 antibody (GE Healthcare) and a colorimetric substrate (TMB, tetramethylbenzidine, Thermo Fischer).

Sequences of positive binders were inserted into the prokaryotic vector pHEN2 (containing 6xHis and cMyc tags) for production in E. coli BL21 (DE3) and purified by conventional immobilized metal ion affinity chromatography.

Apoptosis of human T cells. In vitro apoptosis assays were carried out using the standard in vitro human Jurkat T cell model system, fluorescein isothiocyanate (FITC)-labeled annexin V (Biolegend, San Diego, CA, USA) and propidium iodide (PI). Briefly, recombinant galectin-1 was pre-incubated for 1 h at 4° C. in serum free RPMI 1640 medium before addition to Jurkat cells. The mixture was incubated at 37° C. for 4 h. Cells were then washed once in PBS and once in binding buffer (0.01 M HEPES, 0.14 M NaCl, 2.5 mM CaCl₂, pH 7.4). For staining, cells were incubated for 15 min with FITC-labeled annexin V in the dark at room temperature. The PI (0.25 μg/mL) stain was added to cells just before analysis by flow cytometry.

Microscale thermophoresis (MST. Galectins were labeled using the RED-NHS labeling kit (Nanotemper Technologies, Germany) in accordance with the manufacturer's protocol. Unreacted dye was removed using the provided purification columns. Binding affinity assays were performed in 20 mM Tris-HCl, 150 mM NaCl and 0.1% F127. Samples were loaded into standard Monolith NT.115 capillaries and microscale thermophoresis was measured using a Monolith NT.115 Pico instrument (Nanotemper Technologies, Germany) at ambient temperature. Binding affinities (Kd values) were determined from the fitted curves (three parameter dose-response curve).

ELISA assays. Recombinant human galectins were immobilized in 96-well plates at the indicated concentrations for 16 h at 4° C. After a blocking step with PBS containing 10% (v/v) bovine serum albumin (BSA) (blocking buffer), increasing concentrations of G1N1 were added to each well and incubated for 1 h at room temperature. Binding of G1N1 was revealed using successive incubations with a goat anti-his-tag polyclonal ( 1/1000) antibody (Bio-Rad, ON, Canada) and a donkey anti-goat IgG ( 1/5000) conjugated to horse radish peroxidase (R&D Systems, MN, USA). The colorimetric assay was carried out with TMB (Sigma-Aldrich, MO, USA) according to the manufacturer's recommendations.

Statistical analysis. Statistical significance was evaluated using the unpaired Student's t-test or the Fisher's exact test. Results were considered statistically significant at P≤0.05.

### Example 2: Generation and Production of Galectin-1-Specific VHHs Clones

Following an initial pre-screen round of phage display carried out with GST-biotin to deplete the library from unspecific binders, unbound VHHs were incubated with GAL-1-Biotin beads. A total of three rounds of phage panning were performed. Progressive enrichment during the cycle was validated by measuring the output/input ratio (FIG. 3). A total of 282 VHHs were then picked and analyzed by ELISA for galectin-1 binders. One of the VHHs (clone D06, referred as G1N1) showed strong binding to GAL-1-specific non-adsorbed phage ELISA while the second clone, G01 (G1N2) showed weak binding (FIG. 4). Amino acid sequences of G1N1 and G1N2 are shown in FIG. 5.

### Example 3: Characterization of Galectin-1-Specific VHHs Clones

Both G1N1 and G1N2 were purified by conventional by metal ion affinity chromatography for characterization (FIG. 6). The results showed that the G1N1 clone, but not the G1N2 clone, showed strong and significant inhibition of galectin-1-induced apoptosis of Jurkat T cells (FIG. 7). The inhibition of GAL-1-induced apoptosis was specific as it did not inhibit apoptosis induced by another prototypic galectin, galectin-7 (FIG. 8). The properties of the lead VHH clone G1N1 were further investigated. Binding affinities measurements by microscale thermophoresis (MST) showed that G1N1 binds human and mouse recombinant galectin-1 (which share high sequence identity, as shown in FIG. 9C) in the nanomolar range (FIG. 9B), which is approximately 200-fold higher than the affinity of lactose for galectin-1 (FIG. 9A). MST measurements further showed that G1N1 does not bind to other human or mouse galectins such as galectin-7, human galectin-8, and human galectin-13. The specificity of G1N1 for galectin-1 was confirmed by ELISA testing against galectin-2 (Gal-2), -7 (Gal-7), -13 (Gal-13), and -16 (Gal-16) (FIG. 10A), which provides compelling evidence that the G1N1 antibody binds to an epitope that is present in GAL-1 but is not shared by these other galectins (FIG. 10B).

Although the present invention has been described hereinabove by way of specific embodiments thereof, it can be modified, without departing from the spirit and nature of the subject invention as defined in the appended claims. In the claims, the word “comprising” is used as an open-ended term, substantially equivalent to the phrase “including, but not limited to”. The singular forms “a”, “an” and “the” include corresponding plural references unless the context clearly dictates otherwise.

### REFERENCES

Allo V C, Hauk V, Sarbia N, Pinto N A, Croci D O, Dalotto-Moreno T, Morales R M, Gatto S G, Cocco M N, Stupirski J C, Deladoey Á. Suppression of age-related salivary gland autoimmunity by glycosylation-dependent galectin-1-driven immune inhibitory circuits. Proceedings of the National Academy of Sciences. 2020 Mar. 24; 117(12):6630-9.
Barondes S H, Castronovo V, Cooper D N, Cummings R D, Drickamer K, Feizi T, Gitt M A, Hirabayashi J, Hughes C, Kasai K I. Galectins: a family of animal beta-galactoside-binding lectins. Cell. 1994; 76(4):597-8.
Batzke K, Büchel G, Hansen W, Schramm A. TrkB-target galectin-1 impairs immune activation and radiation responses in neuroblastoma: implications for tumour therapy. International journal of molecular sciences. 2018 March; 19(3):718.
Blanchard H, Bum-Erdene K, Bohar M H, Yu X. Galectin-1 inhibitors and their potential therapeutic applications: a patent review. Expert opinion on therapeutic patents. 2016 May 3; 26(5):537-54.
Blanchetot C, Verzijl D, Mujić-Delić A, Bosch L, Rem L, et al. 2013. Neutralizing nanobodies targeting diverse chemokines effectively inhibit chemokine function. J Biol Chem 288, 25173.
Cedeno-Laurent F, Opperman M, Barthel S R, Kuchroo V K, Dimitroff C J. Galectin-1 triggers an immunoregulatory signature in Th cells functionally defined by IL-10 expression. The Journal of Immunology. 2012 Apr. 1; 188(7):3127-37.
Chakraborty A, Dimitroff C J. Cancer immunotherapy needs to learn how to stick to its guns. The Journal of clinical investigation. 2019 Dec. 2; 129(12):5089-91.
Chen Q, Han B, Meng X, Duan C, Yang C, Wu Z, Magafurov D, Zhao S, Safin S, Jiang C, Cai J. Immunogenomic analysis reveals LGALS1 contributes to the immune heterogeneity and immunosuppression in glioma. International journal of cancer. 2019 Jul. 15; 145(2):517-30.
Cruz E, Kayser V. Monoclonal antibody therapy of solid tumors: clinical limitations and novel strategies to enhance treatment efficacy. Biologics: targets & therapy. 2019; 13:33.
Cummings R D, Liu F T. Galectins. Essentials of Glycobiology. 2nd edition. 2009.
Dalotto-Moreno T, Croci D O, Cerliani J P, Martinez-Allo V C, Dergan-Dylon S, Méndez-Huergo S P, Stupirski J C, Mazal D, Osinaga E, Toscano M A, Sundblad V. Targeting galectin-1 overcomes breast cancer-associated immunosuppression and prevents metastatic disease. Cancer research. 2013 Feb. 1; 73(3):1107-17.
De Genst E, Silence K, Decanniere K, Conrath K, Loris R, Kinne J, Muyldermans S, Wyns L. 2006. Molecular basis for the preferential cleft recognition by dromedary heavy-chain antibodies. Proc Nat Acad Sci 103, 4586.
Grosset A A, Labrie M, Vladoiu M C, Yousef E M, Gaboury L, St-Pierre Y. Galectin signatuares contribute to the heterogeneity of breast cancer and provide new prognostic information and therapeutic targets. Oncotarget. 2016 Apr. 5; 7(14):18183.
Hamers-Casterman C T, Atarhouch T, Muyldermans S, Robinson G, Hammers C, Songa E B, Bendahman N, Hammers R. Naturally occurring antibodies devoid of light chains. Nature. 1993 June; 363(6428):448-8.
Jin Q Y, Li Y S, Qiao X H, Yang J W, Guo X L. Targeting galectins in T cell-based immunotherapy within tumor microenvironment. Life Sciences. 2021 March 27:119426.
Labrie M, De Araujo L O, Communal L, Mes-Masson A M, St-Pierre Y. Tissue and plasma levels of galectins in patients with high grade serous ovarian carcinoma as new predictive biomarkers. Scientific reports. 2017 Oct. 16; 7(1):1-3.
Liu F T, Rabinovich G A. Galectins as modulators of tumour progression. Nature Reviews Cancer. 2005 January; 5(1):29-41.
Liu S D, Whiting C C, Tomassian T, Pang M, Bissel S J, Baum L G, Mossine V V, Poirier F, Huflejt M E, Miceli M C. Endogenous galectin-1 enforces class I-restricted TCR functional fate decisions in thymocytes. Blood, The Journal of the American Society of Hematology. 2008 Jul. 1; 112(1):120-30.
Lykken J M, Horikawa M, Minard-Colin V, Kamata M, Miyagaki T, Poe J C, Tedder T F. Galectin-1 drives lymphoma CD20 immunotherapy resistance: validation of a preclinical system to identify resistance mechanisms. Blood, The Journal of the American Society of Hematology. 2016 Apr. 14; 127(15):1886-95
McMahon C, Baier A S, Pascolutti R, Wegrecki M, Zheng S, et al. 2018. Yeast surface display platform for rapid discovery of conformationally selective nanobodies. Nat Struct Mot Biol 25, 289 Moutel S, Bery N, Bernard V, Keller L, Lemesre E, et al. 2016. NaLi-H1: A universal synthetic library of humanized nanobodies providing highly functional antibodies and intrabodies. Elife 5.
Nambiar D K, Aguilera T, Cao H, Kwok S, Kong C, Bloomstein J, Wang Z, Rangan V S, Jiang D, Von Eyben R, Liang R. Galectin-1-driven T cell exclusion in the tumor endothelium promotes immunotherapy resistance. The Journal of clinical investigation. 2019 Dec. 2; 129(12):5553-67.
Perillo N L, Uittenbogaart C H, Nguyen J T, Baum L G. Galectin-1, an endogenous lectin produced by thymic epithelial cells, induces apoptosis of human thymocytes. The Journal of experimental medicine. 1997 May 19; 185(10):1851-8.
Pinho S S, Reis C A. Glycosylation in cancer mechanisms and clinical implications. Nature Reviews Cancer. 2015 September; 15(9):540-55.
Rabinovich G A, Toscano M A. Turning‘sweet’on immunity: galectin-glycan interactions in immune tolerance and inflammation. Nature Reviews Immunology. 2009 May; 9(5):338-52.
Rodríguez E, Schetters S T, van Kooyk Y. The tumour glyco-code as a novel immune checkpoint for immunotherapy. Nature Reviews Immunology. 2018 March, 18(3):204-11.
Salatino M, Dalotto-Moreno T, Rabinovich G A. Thwarting galectin-induced immunosuppression in breast cancer. Oncoimmunology. 2013 May 1; 2(5):e24077.
Sundblad V, Morosi L G, Geffner J R, Rabinovich G A. Galectin-1: a jack-of-all-trades in the resolution of acute and chronic inflammation. The Journal of Immunology. 2017 Dec. 1; 199(11):3721-30.
Tang D, Gao J, Wang S, Yuan Z, Ye N, Chong Y, Xu C, Jiang X, Li B, Yin W, Miao Y. Apoptosis and anergy of T cell induced by pancreatic stellate cells-derived galectin-1 in pancreatic cancer. Tumor Biology. 2015 July; 36(7):5617-26.
Than N G, Balogh A, Romero R, Kárpáti É, Erez O, Szilágyi A, Kovalszky I, Sammar M, Gizurarson S, Matkó J, Závodszky P. Placental protein 13 (PP13)-a placental immunoregulatory galectin protecting pregnancy. Frontiers in immunology. 2014 Aug. 20; 5:348.
Van Woensel M, Mathivet T, Wauthoz N, Rosiére R, Garg A D, Agostinis P, Mathieu V, Kiss R, Lefranc F, Boon L, Belmans J. Sensitization of glioblastoma tumor micro-environment to chemo- and immunotherapy by Galectin-1 intranasal knock-down strategy. Scientific reports. 2017 Apr. 27; 7(1):1-4.
Verschuere T, Toelen J, Maes W, Poirer F, Boon L, Tousseyn T, Mathivet T, Gerhardt H, Mathieu V, Kiss R, Lefranc F. Glioma-derived galectin-1 regulates innate and adaptive antitumor immunity. International journal of cancer. 2014 February; 134(4):873-84.
Wu A A, Drake V, Huang H S, Chiu S, Zheng L. Reprogramming the tumor microenvironment: tumor-induced immunosuppressive factors paralyze T cells. Oncoimmunology. 2015 Jul. 3; 4(7):e1016700.
Salema et al., MAbs vol. 8, No. 7, 1286-1301.
Gene, R W et al., (2015) J Immunol Methods 416, 29-39.
Kumaran, J. (2012). Methods Mol Biol 911, 105-124.
Cousin and Cloninger, Int J Mol Sci. 2016 September; 17(9): 1566.
Sundblad et al., J Immunol Dec. 1, 2017, 199 (11) 3721-3730.

1. A monovalent antibody that specifically binds to human galectin-1 (hGAL-1) and inhibits its activity, wherein the monovalent antibody comprises the following combination of complementarity determining regions (CDRs): a CDR1 comprising an amino acid sequence having at least 80% identity with the sequence STFSEDA (SEQ ID NO:1); a CDR2 comprising an amino acid sequence having at least 80% identity with the sequence GFANPHS (SEQ ID NO:2); and a CDR3 comprising an amino acid sequence having at least 80% identity with the sequence ASHKKTRAPAATFET (SEQ ID NO:3).

2. (canceled)

3. The monovalent antibody of claim 1, which comprises one of the following combinations of CDRs: a CDR1 comprising the sequence STFSEDA (SEQ ID NO:1); a CDR2 comprising the sequence GFANPHS (SEQ ID NO:2); and a CDR3 comprising the sequence ASHKKTRAPAATFET (SEQ ID NO:3).

4. The monovalent antibody of claim 1, wherein the monovalent antibody is a single-domain antibody.

5. The monovalent antibody of claim 1, which comprises:
(i) a framework region (FR) 1 comprising an amino acid sequence having at least 50% identity with the sequence MAEVQLQASGGGFVQPGGSLRLSCAASG (SEQ ID NO:4);
(ii) a FR2 comprising an amino acid sequence having at least 50% identity with the sequence MGWFRQAPGKEREFVSAIS (SEQ ID NO:5);
(iii) a FR3 comprising or consisting of an amino acid sequence having at least 50% identity with the sequence YYADSVKGRFTISRDNSKNTVYLQMNSLRAEDTATYYCA (SEQ ID NO:6);
(iv) a FR4 comprising an amino acid sequence having at least 50% identity with the sequence YWGQGTQVTVSS (SEQ ID NO:7); or
(v) any combination of (i) to (iv).

6. (canceled)

7. The monovalent antibody of claim 1, which comprises:
(i) a FR1 comprising the sequence MAEVQLQASGGGFVQPGGSLRLSCAASG (SEQ ID NO:4);
(ii) a FR2 comprising the sequence MGWFRQAPGKEREFVSAIS (SEQ ID NO:5);
(iii) a FR3 comprising the sequence YYADSVKGRFTISRDNSKNTVYLQMNSLRAEDTATYYCA (SEQ ID NO:6);
(iv) a FR4 comprising the sequence YWGQGTQVTVSS (SEQ ID NO:7); or
(v) any combination of (i) to (iv).

8. The monovalent antibody of claim 1, comprising an amino acid sequence having at least 80% identity with the sequence set forth in SEO ID NO:8.

9. (canceled)

10. The monovalent antibody of claim 8, comprising the amino acid sequence set forth in SEQ ID NO:8.

11. The monovalent antibody according to claim 1, wherein said monovalent antibody is fused to at least one antibody constant domain, or a fragment thereof.

12. (canceled)

13. The monovalent antibody according to claim 11, wherein the fragment comprises a CH2 domain and CH3 domain of a human antibody.

14. The monovalent antibody of claim 1, wherein said antibody is conjugated to a label, a nanoparticle, a drug, a peptide, a nucleic acid, a toxin, an enzyme, a radioisotope, or a half-life extending moiety.

15. A nucleic acid comprising a nucleotide sequence encoding the monovalent antibody defined in claim 1.

16-19. (canceled)

20. A pharmaceutical composition comprising the monovalent antibody defined in claim 1, or a nucleic acid encoding the monovalent antibody, and one or more pharmaceutically acceptable carriers, excipient, and/or diluents.

21. A method for binding human galectin-1 (hGAL-1) comprising contacting said hGAL-1 with the monovalent antibody of claim 1.

22. The method of claim 21, wherein said hGAL-1 is expressed at the surface of a cell.

23. A method for inhibiting galectin-1-mediated apoptosis in a cell, said method comprising contacting said cell with an effective amount of the monovalent antibody of claim 1.

24. (canceled)

25. The method of claim 23, wherein said cell is a T lymphocyte.

26-27. (canceled)

28. A method for treating a galectin-1-expressing cancer in a subject, said method comprising administering to said subject an effective amount of the monovalent antibody of claim 1, or a nucleic acid encoding said monovalent antibody.

29. (canceled)

30. The method of claim 28, wherein the cancer is a breast cancer, a melanoma, an ovarian cancer or a lymphoma.

31. (canceled)

32. A method for treating a disease or condition associated with pathological neovascularization or angiogenesis in a subject comprising administering to said subject an effective amount of the monovalent antibody of claim 1, or a nucleic acid encoding said monovalent antibody.

33. A method for inhibiting tissue or organ fibrosis in a subject comprising administering to said subject an effective amount of the monovalent antibody of claim 1, or a nucleic acid encoding said monovalent antibody.

34-72. (canceled)
